(19) United States
(12) Patent Application Publication
(10) Pub. No.: US 2009/0216189 A1
(43) Pub. Date: Aug. 27, 2009

(54) DEVICE AND METHOD FOR CONTROLLING FLOW OF LIQUID FLUID IN ARTHROSCOPIC SURGERY

(76) Inventors: Seth Gasser, Tampa, FL (US); Sergio Gutierrez, Tampa, FL (US)

Correspondence Address: SCHOX PLC, 730 Florida Street #2, San Francisco, CA, 94110, US

(21) Appl. No.: 12/372,966

(22) Filed: Feb. 18, 2009

Related U.S. Application Data
(63) Continuation-in-part of application No. 11/860,036, filed on Sep. 24, 2007.

(51) Int. Cl. A61M 1/00; B65B 35/00

(52) U.S. Cl. 604/119; 53/396

(57) ABSTRACT

In the preferred embodiment, the invention is a device for foot actuated fluid flow control through a surgical site in arthroscopic surgery. The device preferably includes: a foot pedal and a fluid regulator. Compression of the foot pedal actuates the fluid regulator, which switches the fluid regulator to the OPEN state, preferably in a variable fashion. The device also includes tubing that allows the device to be connected and used with a variety of existing systems for arthroscopic surgery.

This application is a continuation-in-part of prior application Ser. No. 11/860,036, filed 24-Sep.-2007, which is incorporated in its entirety by this reference.

### TECHNICAL FIELD

This invention relates generally to the surgical field, and more specifically to a new and useful device and method for controlling the flow of sterile fluid through the surgical site in the arthroscopic surgical field.

### BACKGROUND

Arthroscopes and other optical instruments such as endoscopes are in common use by orthopedic surgeons because they enable minimally invasive surgery. For example, the use of such instruments typically reduces the amount of time required to perform a surgical operation, reduces the patient's recovery time, reduces the post-operative pain experienced by patients, and reduces the costs compared with traditional surgery. Moreover, the surgery can often be handled on an outpatient basis.

In arthroscopic surgery, an arthroscope is used to assist in the visual inspection of a joint such as a knee or shoulder joint. The procedure begins with an incision into the joint, and sterile fluid, typically saline, is introduced into the joint space to provide a better view. The arthroscope is then inserted and the inside of the joint is viewed by displaying the image on a monitor. During the procedure, tissue and other debris accumulate in the fluid at the site of the procedure and interfere with the field of vision. To clear the view, the fluid at the site is purged and additional sterile fluid is delivered to the site. A fluid circuit is maintained with sterile fluid provided by a remote reservoir in fluid communication with an inlet of the scope. The fluid is thus delivered from the reservoir to the field of view. The circuit further includes an outlet from the field of view for purging the discharge fluid to a remote receptacle or collection tank for discard. The system typically operates under suction or negative pressure with control provided by a stopcock or other hand-controlled valve assembly downstream of the field of view. When the valve is closed, the fluid is statically maintained within the site of the procedure. The surgeon manually opens the stopcock to flush the site and deliver additional fluid. The suction or negative pressure moves the fluid from the site to the collection tank and delivers fresh fluid from the reservoir of sterile fluid. The surgeon closes the valve to stop the flow when the site is sufficiently flushed. The focus of the arthroscope is then typically readjusted and the procedure is continued.

Current instrumentation for arthroscopy includes at least three hand-actuated devices that the surgeon cannot manipulate simultaneously. The devices are a camera, at least one surgical tool, and the stopcock. During the procedure, the surgeon typically operates the camera with one hand and manipulates the surgical instruments, such as a shaver or biter, with the other hand. To operate the stopcock or other hand-controlled valve assembly, the surgeon must put down either the camera or the surgical instrument to free a hand. If the inlet stopcock is left open, the opening of the outlet stopcock purges the field of view of the fluid to be discarded and delivers sterile fluid to the surgical site. The surgeon must then manually close the outlet stopcock and retrieve the surgical instrument. A typical surgical procedure may require about thirty of such purges, causing the surgeon to have to put down and pick up the surgical instrument that many times. Such action diverts the surgeon's attention from the field of view each time a purge is required and increases the time required to complete the procedure.

Thus, there is a need in the field to create a new and useful device and method that allows the surgeon to purge the field of view and control fluid flow without requiring the surgeon to swap tools in and out of their hands and to divert attention. This invention provides such new and useful device and method.

### BRIEF DESCRIPTION OF THE FIGURES

FIG. 1 is a diagrammatic view of an illustrative embodiment of the invention.

FIG. 2 is a diagrammatic view of an illustrative embodiment of the invention with internal components visible.

FIG. 3 is a diagrammatic view of an illustrative embodiment of the invention in a preferred usage scenario;

FIG. 4 is a diagrammatic view similar to that of FIG. 1 but with the reservoir and collection tank omitted;

FIGS. 5A, 5B, 5C, and 5D are perspective views of the first, second, third, and fourth embodiments, respectively, of the foot pedal;

FIGS. 6A, 6B, and 6C are perspective views of the first, second, and third embodiments, respectively, of the underside of the foot support;

FIGS. 7A, 7B, are 7C are perspective views of the first, second, and third embodiments, respectively, of the foot pedal base;

FIG. 8A is a longitudinal sectional view depicting the valve assembly that is opened and closed by the novel foot pedal;

FIG. 8B is a perspective view of the valve assembly of FIG. 6A;

FIG. 8C is an elevation view depicting the valve assembly of FIGS. 6A and 6B when installed in the base of the novel foot pedal;

FIGS. 9A, 9B, and 9C are diagrammatic top plan, side, and front elevation views, respectively, of a second embodiment of the valve assembly;

FIGS. 10A and 10B are perspective and side views, respectively, of the third embodiment of the invention;

FIG. 11A is a perspective view of the third embodiment with the foot support removed;

FIG. 11B is a perspective view similar to FIG. 11A but with the lever and related parts removed; and

FIG. 11C is a top plan view of the parts depicted in FIG. 11B.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

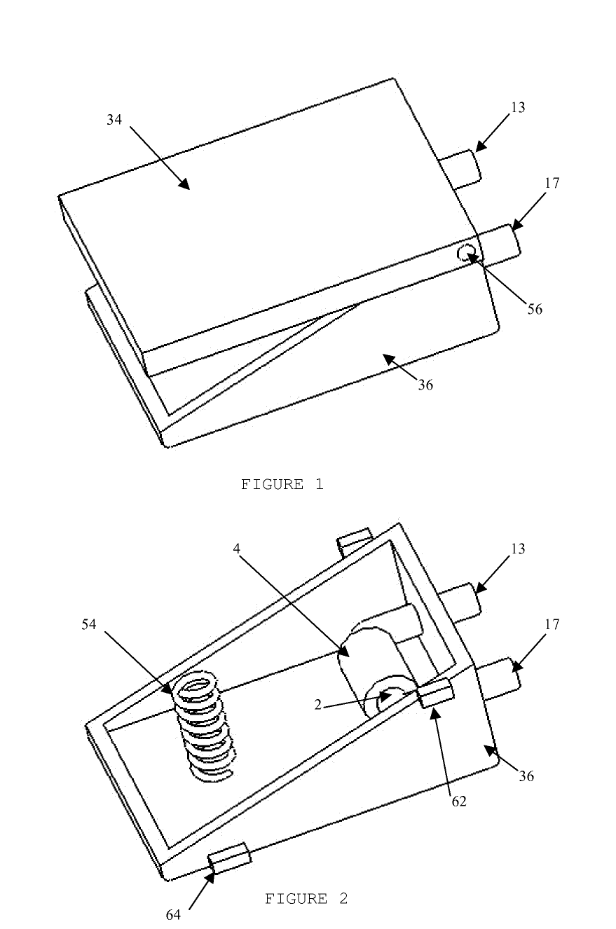

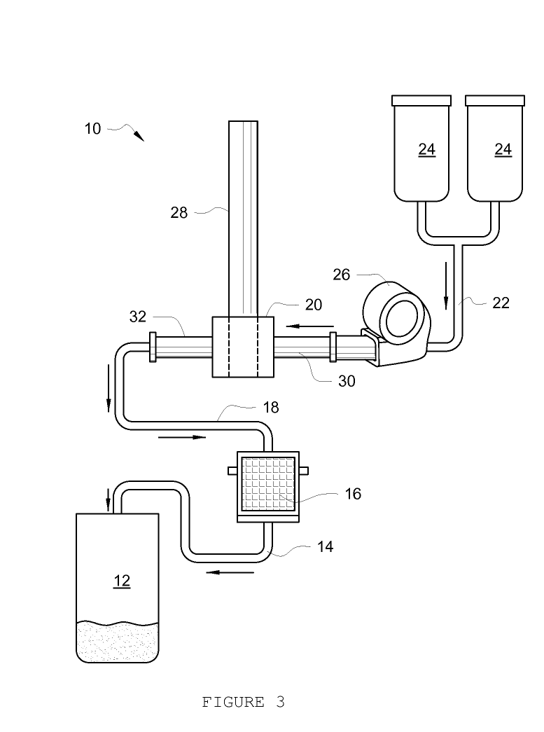

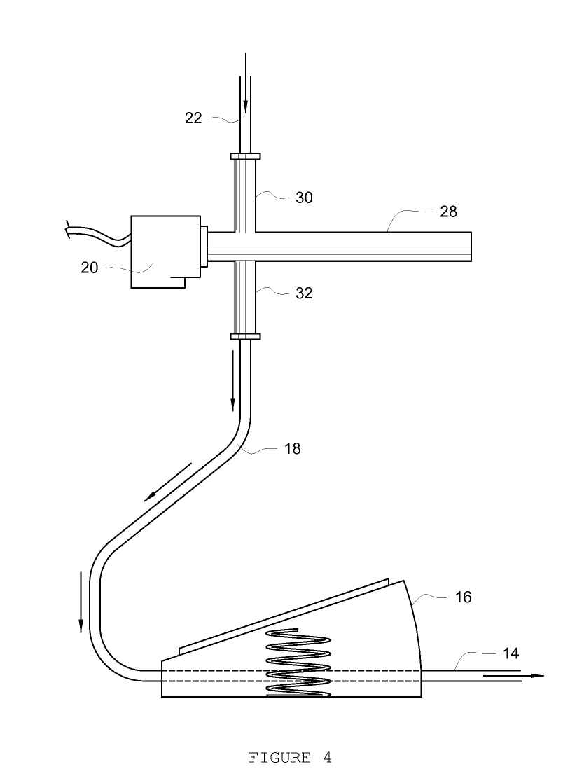

As shown in FIGS. 1-4, the device of the preferred embodiments includes a foot pedal 16 and a fluid regulator 2. The fluid regulator 2 may also include a fluid regulator switch 4 that may electronically actuate the fluid regulator 2 remotely. The device may also include tubing (outlet hose 14 and inlet hose 18) that allows the device to be connected to existing systems for arthroscopic surgery that include arthroscopic surgical tools (contained within sheath 28), an arthroscopic camera 20, sterile fluid (such as saline), a pump 26, sterile tubing 22, a sterile fluid reservoir 24, and a waste fluid collection tank 12. As shown in FIGS. 3 and 4, flow of the sterile fluid preferably originates from the fluid reservoir 24, travels through sterile tubing 22 to the surgical site, then travels from the surgical site to the collection tank 12. The device preferably allows this fluid path to be controlled by the actuation of the foot pedal 16 by the surgeon's foot, allowing his hands to be fully dedicated to controlling the arthroscopic surgical tools and the arthroscopic camera 20, thus increasing efficiency in the surgical procedure. A surgeon will typically rest his or her foot on or near foot pedal 16 during the surgical procedure and will begin depression of the pedal with a foot by applying pressure to the foot support 34 when the surgeon's field of vision is obscured.

The foot pedal 16 preferably functions to actuate the fluid regulator 2 (either directly or indirectly through a fluid regulator switch 4) and secures and supports the fluid regulator 2 and/or the fluid regulator switch 4. The foot pedal 16 includes a foot support 34 and a stationary base 36. The foot pedal 16 also includes a spring 54 to bias the foot pedal 16 in an extension direction, a hinge pin 56 (located preferably at the end of the foot pedal 16 distal to the user, but alternatively at the end of the foot pedal 16 proximal to the user) that functions to connect the foot pedal 34 and the stationary base 36 and to allow the foot support 34 to rotate about the hinge pin 56 when compressed by the user, an extension limiter 62 to prevent over-extension of the foot pedal 16 by the spring 54, and a compression limiter 64 to prevent over-compression of the foot pedal 16 by the user. If the fluid regulator 2 is fully secured and supported by the foot pedal 16, the foot pedal may also include an OUT connection 13 to the outlet of the fluid regulator 2 that allows the outlet hose 14 to connect with the outlet of the fluid regulator 2 and an IN connection 17 to the inlet of the fluid regulator 2 that allows the inlet hose 17 to connect with the inlet of the fluid regulator 2. The axis of the OUT connection 13 and the IN connection 17 are preferably parallel to each other and perpendicular to the axis of the hinge pin 56 as shown in FIGS. 1 and 2. The OUT connection 13 and IN connection 17 are also preferably positioned at the distal end of the foot pedal 16, i.e., in a location remote to the toes of the user. This arrangement reduces the clutter of the lines around the user's feet and therefore provides a greater degree of safety. Alternatively, the axis of the OUT connection 13 may be collinear with the axis of the IN connection 17 and parallel to the axis of the hinge pin 56 to facilitate manufacturing and assembly of the device.

The foot pedal 16 preferably also includes a tint of a color that functions to aid the surgeon in visually differentiating the foot pedal 16. In many systems for arthroscopic surgery, multiple foot pedals exist near the surgeon's foot, each controlling a different element of the system, for example, various surgical tools. Because of this, being able to differentiate one foot pedal from another easily will increase the efficiency of the surgical procedure. Alternatively, the foot pedal 16 may also be differentiated based upon physical features such as shape, size, texture, and material. For example, the surgeon will most likely be concentrating on the monitor where he or she can view the procedure through the arthroscopic camera. For this reason, a method of differentiation that allows the surgeon to continue to view the monitor while correctly locating the foot pedal 16 will improve efficiency in the surgical procedure. In differentiation based upon size and shape, for example, the foot pedals generally installed to be used in systems for arthroscopic surgeries are large and made of metal and covered in rubber appropriate for sterilization. The foot pedal 16 of the preferred embodiments is preferably relatively smaller than the existing system foot pedals and preferably is of a shape different from those foot pedals already in use. The foot pedal 16 may also be of a different material, such as plastic to differentiate from the metal and rubber foot pedals of the system. Rough or ridged surface textures that may be felt through the surgeon's shoe as they feel for the foot pedal 16 may also be used. The texture may also function to increase friction with the surgeon's shoe that can be easily detected. Alternatively, sounds such as a bell or buzzer to indicate when the surgeon has touched the foot support 24 of foot pedal 16 or sound resulting from the surgeon's shoe rubbing against the texture 35 may also be used to differentiate the foot pedal 16. However, any other suitable material or method of differentiation of foot pedal 16 may be used.

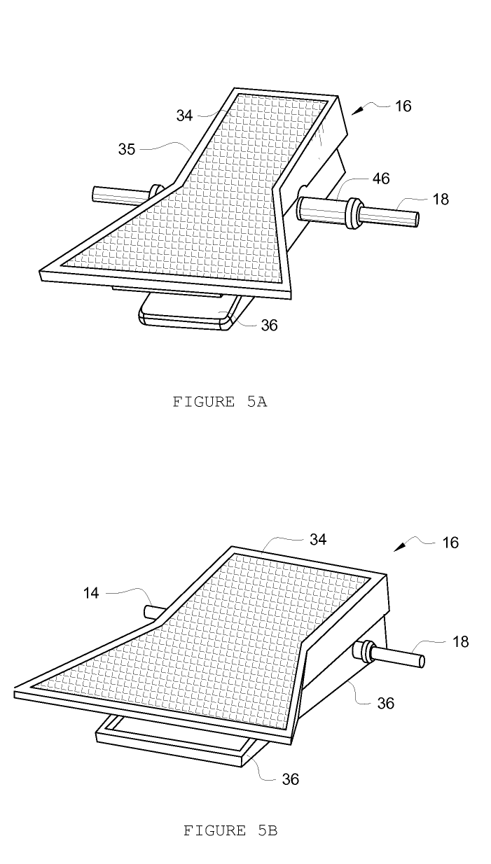

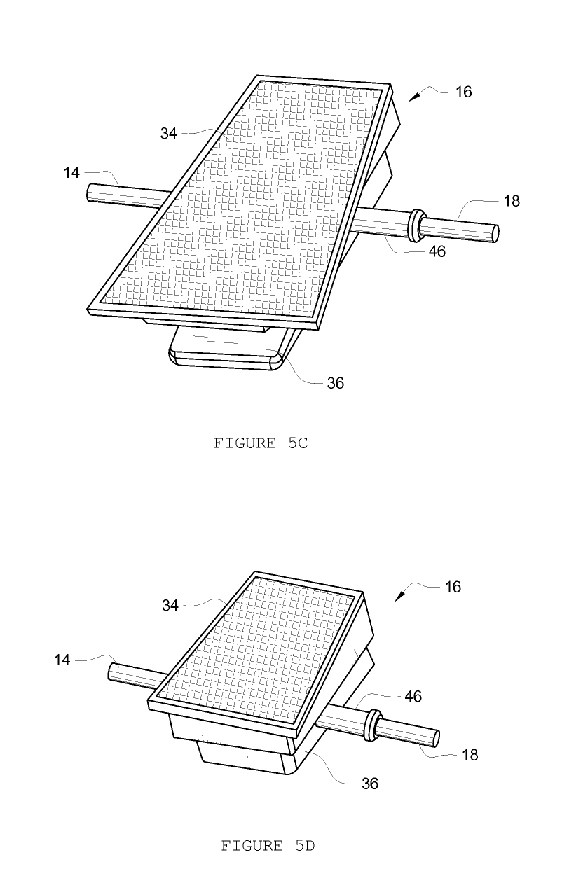

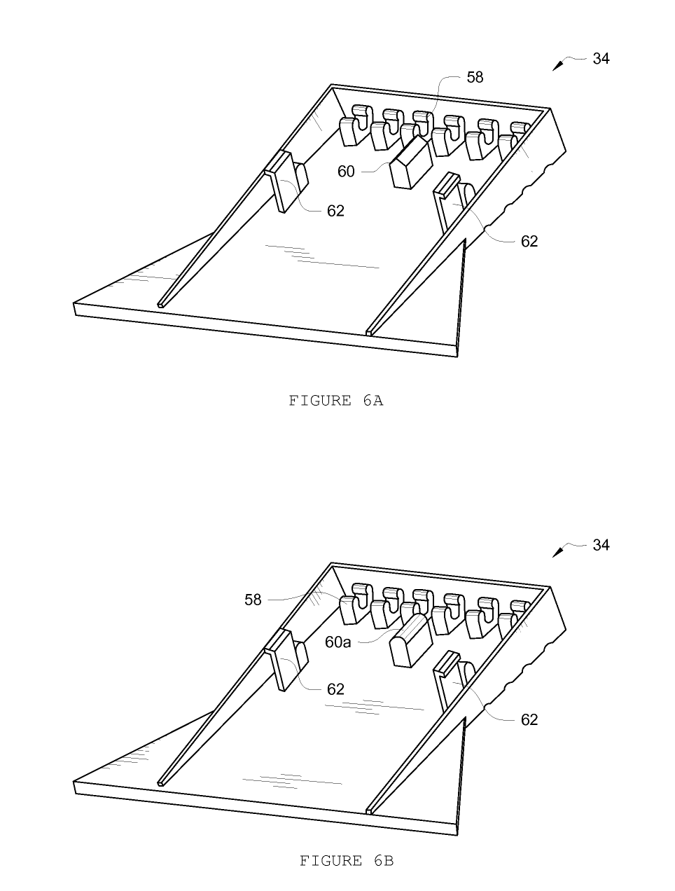

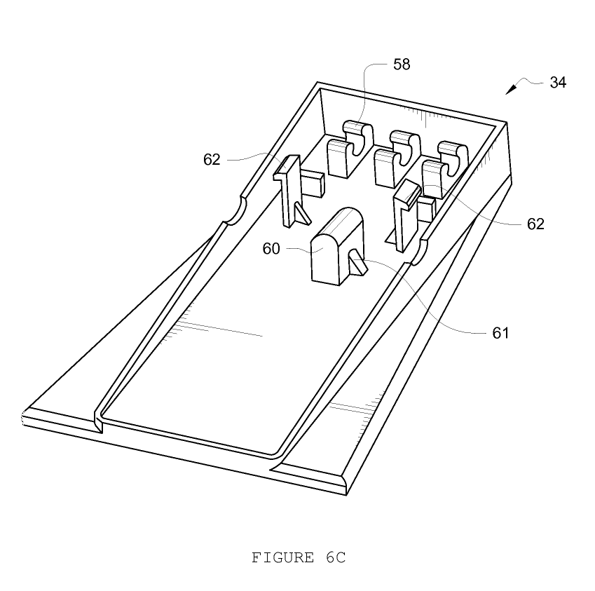

As shown in FIGS. 5A to 5D, the foot support 34 may also include a textured surface 35. The textured surface 35 of foot support 34 functions to supply a high coefficient of friction to enable rocking motion of foot support 34 about the hinge pin 56 and base 36 in the substantial absence of foot slippage. A preferred embodiment of the geometry of the foot pedal 16 is shown in FIG. 5A. The width of foot support 34 is increased in FIG. 5B relative to the width thereof in FIG. 5A, increasing the resting surface for the user's foot. A variation to the geometry of the foot pedal 16 is shown in FIG. 5C. The increased surface area of the foot support 34 of FIG. 5C facilitates placement of the user's foot. As shown in FIG. 5D, the overall size of the foot pedal 16 of the variation shown in FIG. 5C enables the placement of foot pedal 16 in tighter spaces.

The foot pedal 16 is preferably made of reasonably inexpensive components and materials, thus making it suitable for disposal following a single surgical procedure. The foot support 34 and the base 36, each with features for extension limiter 62, compression limiter 64, hinge pin 56, spring 54, and other features to secure and support the fluid regulator 2 and/or fluid regulator switch 4, are preferably manufactured using moldable plastic to allow manufacture by injection molding, a relatively inexpensive method of mass production. The foot pedal 16 is preferably a relatively simple assembly of the foot support 34 (with features built in), base 36 (with features built in), hinge pin 56, and spring 54. The hinge pin 56 may alternatively be a feature integrated into foot support 34 and base 36. However, any other suitably inexpensive material and manufacturing method may be used. Alternatively, the foot pedal 16 may be of a material that may be sterilized to be reused, for example, stainless steel.

The fluid regulator 2 preferably functions to block the flow of the sterile fluid when closed and to allow the flow of the sterile fluid when open. The foot pedal 16 preferably actuates the fluid regulator 2. The fluid regulator 2 is preferably normally in a CLOSED state and switched to an OPEN state upon actuation from the foot pedal 16. The OPEN state of the fluid regulator 2 is preferably variable, for example, the OPEN state may allow a low flow, high flow, or a flow type in between low and high. For example, if input from the user through the foot pedal 16 indicates a medium flow, the fluid regulator 2 is preferably switched to a partially OPEN state. The fluid regulator 2 is preferably mechanically actuated from the CLOSED state to the OPEN state, but may also alternatively include a fluid regulator switch 4 that is actuated by the foot pedal 16 and electronically switches the fluid regulator 2 from the CLOSED state to the OPEN state. Alternatively, the fluid regulator 2 may have one CLOSED state and one OPEN state.

The outlet hose 14 and the inlet hose 18 are preferably able to connect with a variety of existing systems for arthroscopic surgery. For example, the outlet hose 14 and the inlet hose 18 may be of an elastic material of a diameter that can accommodate to a variety of sizes of connectors to the systems for arthroscopic surgery. For example, given a range of sizes of connections to such systems, the diameter for the outlet hose 14 and inlet hose 18 is chosen such that the elasticity of the material can allow the tubing to remain securely fastened to a connector of the small variety as well as accommodate a connector of the large variety. Alternatively, adapters that are appropriate for connection to outlet hose 14 or inlet hose 18 on one end and fully accommodated for connection to a specific type of system on the other end may be provided to allow the device to connect reliably to a variety of systems. A series of different outlet hose 14 and inlet hose 18 that are appropriate for connection to the OUT connection 13 and the IN connection 17 respectively on one end and fully accommodated for connection to a specific type of system on the other end may also be provided as an alternative.

The foot pedal 16, the fluid regulator 2, and the tubing are preferably single use. In embodiments that include the fluid regulator switch 4, the fluid regulator 2 may be intended for single use, while fluid regulator switch 4 may be intended to be reused. Alternatively, the foot pedal 16 may be sterilized to be reused and a new fluid regulator 2 may be used after each surgical procedure. However, any other suitable combination of intended single use or intended reuse suitable for the procedure may be used.

The disposable foot pedal 16, fluid regulator 2, outlet hose 14, and inlet hose 18 are preferably sold to hospitals or any other institutions with systems for arthroscopic surgery installed and in use. The foot pedal 16, fluid regulator 2, outlet hose 14, and outlet hose 18 are preferably each sterilized and then placed into a sterile vacuum pouch and sold as a group. The foot pedal 16 and fluid regulator 2 may be packaged into the same sterilized package and sold along with a sterilized package containing the outlet hose 14 and the inlet hose 18. The foot pedal 16 and fluid regulator 2 are preferably pre-assembled prior to packaging and sold as a single ready to use unit. The user will need to assemble the tubing to the assembled foot pedal unit and attach the tubing to the system (or to an adapter and then to the system) for use in surgery. This packaging arrangement is most useful in the case of a series of different outlet hoses 14 and inlet hoses 16 that have one end adapted to fully accommodate a particular type of system. This allows the assembled foot pedal unit to be purchased in bulk and the tubing for the particular machines that the user desires to use with the device to be purchased separately. Alternatively, the foot pedal 16, fluid regulator 2, outlet hose 14, and inlet hose 18 may be packaged into the same sterilized package. In this variation, the foot pedal 16, fluid regulator 2, outlet hose 14, and inlet hose 18 are preferably pre-assembled prior to packaging and sold as a single ready to plug-in unit. The user will need to attach the tubing to the system (or to an adapter and then to the system) for use in surgery. If adapters are used to fully accommodate to the system in use, the adapters are preferably sterilized and packaged in separate packaging from the assembled foot pedal unit and the tubing, allowing the hospital or institution to purchase the foot pedal 16, fluid regulator 2, outlet hose 14, and outlet hose 18 in bulk and purchase the adapters for the machines that they desire to use the device with separately.

The following preferred embodiments preferably include all of the features and elements described above.

### I. First Embodiment

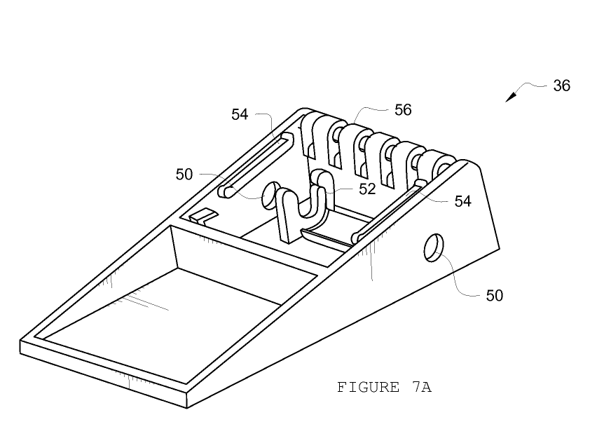

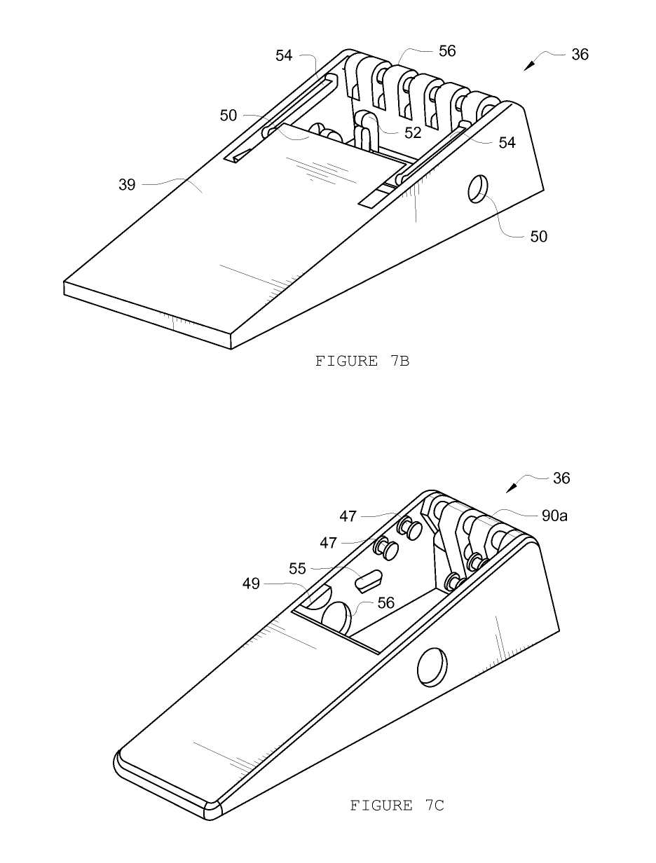

As shown in FIGS. 6A to 6C, 7A to 7C, and 8A to 8C, the first preferred embodiment of the invention includes a check ball 38 within a pliable annular valve seat 40, both assembled into a single full valve assembly 39 as the fluid regulator 2 and a foot pedal 16 that functions to actuate the pliable annular valve seat 40 and secure and support the full valve assembly. The first preferred embodiment also includes rubber hose attachments 46 and 48 as the OUT connection 13 and the IN connection 17 respectively.

The underside of a variation of the foot support 34 of the first preferred embodiment is shown in FIG. 6A. The foot support 34 of this variation includes a socket 58 that is formed integrally with the foot support 34 and functions to snugly and rotationally receive hinge pin 56 of base 36 to provide the pivotal or rocking motion between foot support 34 and base 36, a fluid regulator actuator 60 that is formed integrally with foot support 34 and functions to actuate the pliable annular valve seat 40 (further explained below), and a pair of extension limiters 62 that are also formed integrally with the foot support 34 and function to prevent over-extension of the foot pedal 16.

The underside of an alternative variation of the foot support 34 is shown in FIG. 6B. In this variation, the fluid regulator actuator 60a is an alternative to fluid regulator actuator 60 and is shaped to prevent over-compression during actuation of the pliable annular valve seat 40. Over-compression of the pliable annular valve seat 40 may result in stopping the flow of the sterile fluid.

The underside of a variation of the foot support 34 shown in FIG. 6A is shown in FIG. 6C. This variation of the foot support 34 further includes supports 61 that strengthen the extension limiters 62 and fluid regulator actuator 60.

A variation of the base 36 of the first preferred embodiment is shown in FIG. 7A. The base 36 of this variation includes openings 50 to accommodate the respective ends of rubber hose attachments 46 and 48, transversely spaced apart clips 52 that are formed integrally with base 36 and function to secure and support the full valve assembly, springs 54 positioned on the interior of the opposing vertical sidewalls of the base 36 that extend the foot pedal 16 and return foot support 34 to its position of repose when the surgeon's foot is separated from said foot support, and hinge pin 56 that is formed integrally with base 38 at its highest end.

A variation of the base 36 shown in FIG. 7A is shown in FIG. 7B. The base 36 of this variation of the first preferred embodiment further includes a cover 39 that covers the open lower end of the base 36, leaving clips 52 and hinge pin 56 uncovered. The opposing sides of the base are also embossed “IN” and “OUT” to visually indicate the inlet and outlet of the full valve assembly 39 respectively adjacent to the openings 50.

Another variation of the base 36 of the first preferred embodiment is shown in FIG. 7C. The base 36 of this variation has a relatively narrow structure and is used with a narrow foot support, such as depicted in FIG. 6C. Base 36 includes a pair of knobs 47 that extend into the hollow interior of base 36 from its opposing vertical sidewalls. Rubber bands (not shown) are secured to said knobs 47 and function as the springs 54 to extend the foot pedal 16 and return foot support 34 to its position of repose when the surgeon's foot is separated from said foot support 34. Base 36 further includes retaining clips 49 (not shown in drawing) that function to secure and support the 6 and are positioned adjacent to openings 50 that accommodate the outlet rubber hose 48 and inlet rubber hose 48. Base 36 further includes extension limiter interface 55 that interfaces with extension limiters 62 to prevent the over-extension of the foot pedal 16 by the springs 54.

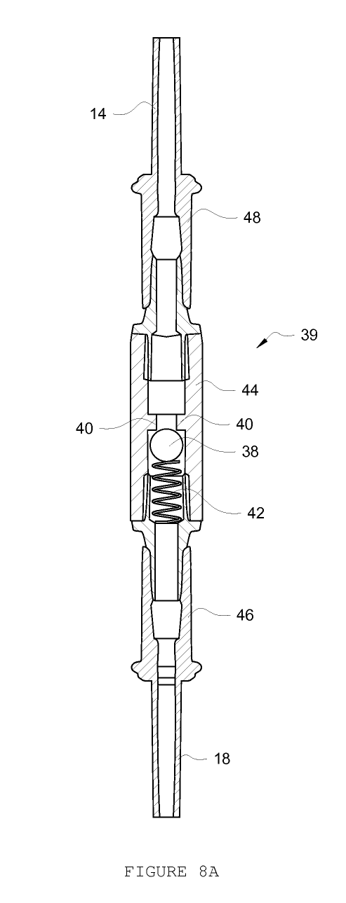

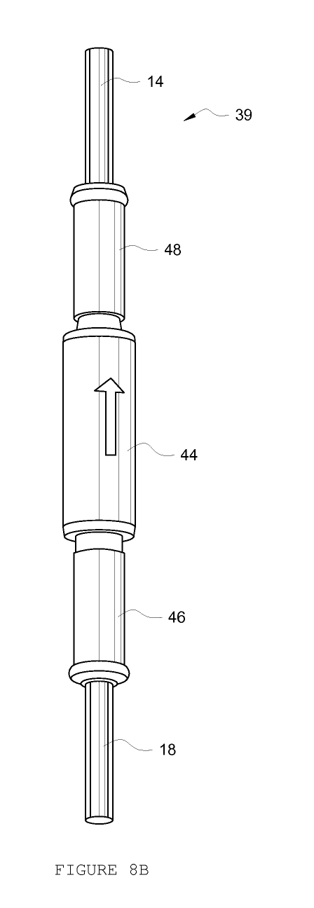

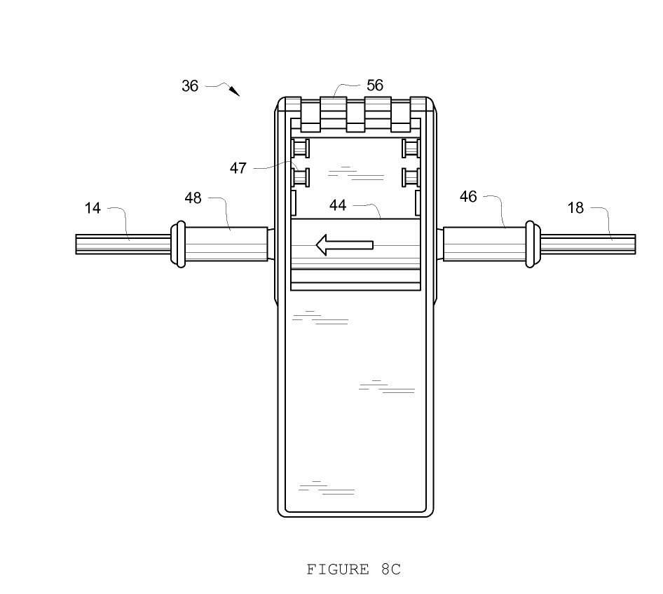

As shown in FIGS. 8A and 8B, the full valve assembly 39 includes check ball 38 held against the pliable annular valve seat 40 by spring 42. Radially inward-extending valve seat 40 is formed integrally with rubber tubing 44. Check ball 38 and spring 42 are positioned within the lumen of said rubber tubing 44. The length of rubber tubing 44 is substantially equal to the width of base 36. The outlet rubber hose attachment 46 abuts a first end of rubber tubing 44 and the inlet rubber hose attachment 48 abuts an opposite, second end of rubber tubing 44. Moreover, inlet rubber hose attachment 46 ensleeves inlet hose 18 and outlet rubber hose attachment 48 ensleeves outlet hose 14. As shown in FIG. 8C, the full valve assembly 39 is assembled into the base 36 through openings 50 and secured and supported using clips 52. When the foot pedal 16 is compressed, valve actuator 60 or 60a bears against rubber tubing 44 and subsequently distorting pliable annular valve seat 40 into an oblong or other non-annular configuration so that liquid fluid leaks around spherical ball valve 38. The distortion of the pliable annular valve seat 40 allows for variable OPEN states upon compression of the foot pedal 16, allowing the surgeon to compress the foot pedal 16 less for low flow, compress the foot pedal 16 more for high flow, or compress the foot pedal 16 anywhere in between for flow types in between low and high.

### 2. Second Preferred Embodiment

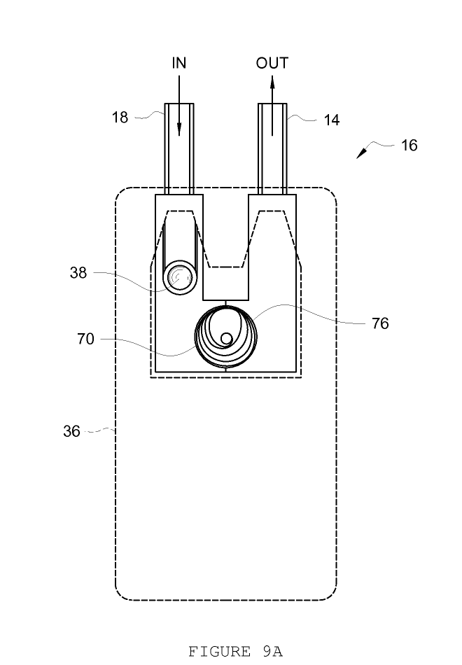

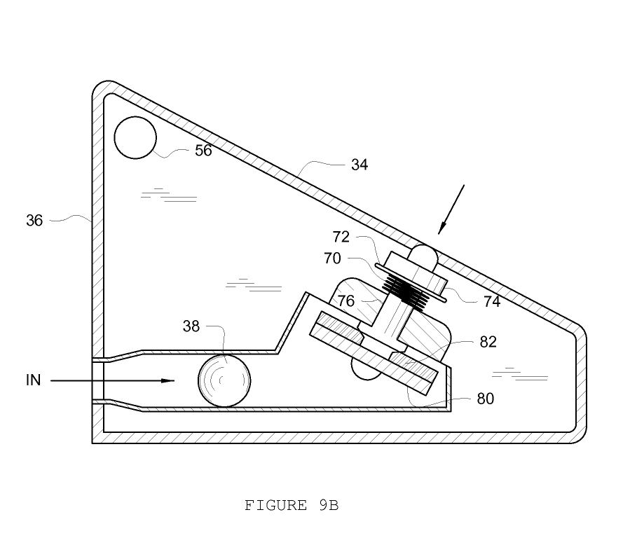

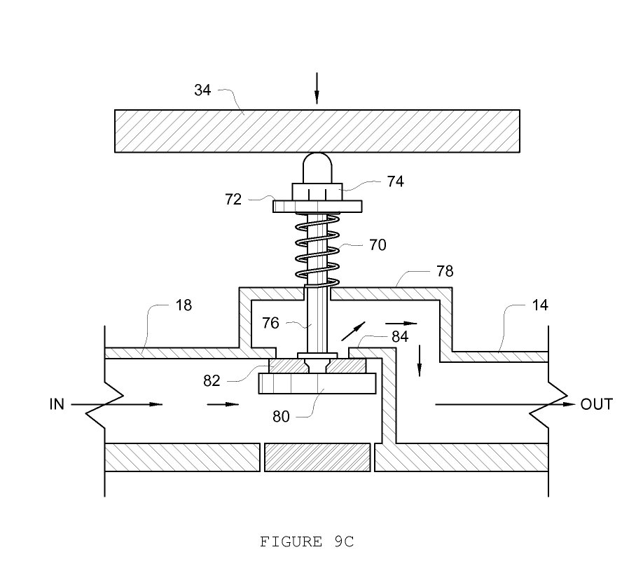

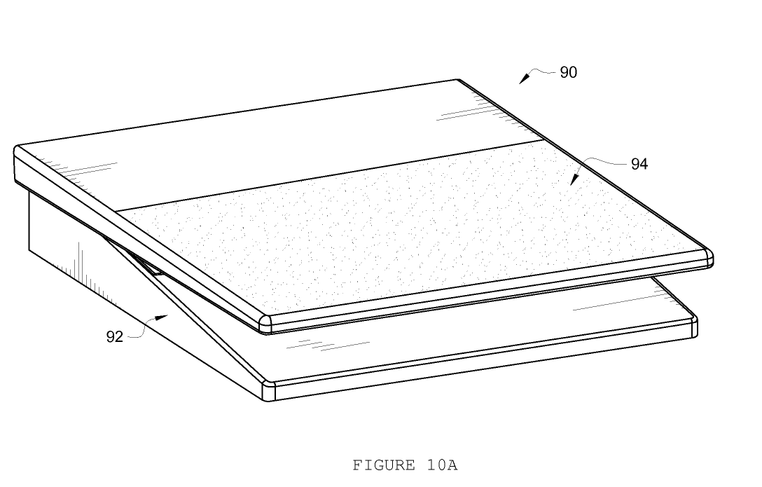

As shown in FIGS. 9A to 9C, the second preferred embodiment of the invention includes a check valve 80, a rubber o-ring 82 over an annular valve seat 84, a spring 70, a washer 72, a nut 74, and a post 76 inserted into boss 78 that are assembled as the as the fluid regulator 2.

As perhaps best understood in connection with FIG. 9C, spring 70 urges washer 72 upwardly. Nut 74 limits the maximum expansion of said spring 70. Spring 70 ensleeves post 76, which is supported by boss 78. Post 76 is displaced downwardly by the foot support 34 when the foot pedal 16 is compressed, thereby loading spring 70. Check valve 80, sealed by rubber O-ring 82, is unseated from annular valve seat 84 when the post is displaced downward, thereby allowing sterile fluid to flow from reservoir 24 to collection tank 12 as indicated by the plurality of flow arrows.

The check valve 80 and rubber o-ring 82 may also function as extension limiters 62. The check valve 80 may additionally also function as the compression limiter 64 when the post is depressed downward, causing the check valve 80 to move downward until the check valve 80 touches the bottom surface. Once the bottom surface is reached, the foot pedal 16 may not be compressed any further.

The foot pedal 16 of the second preferred embodiment is preferably similar or identical to those described in the first preferred embodiment. Alternatively, depression of the post 76 may be accomplished by a flat surface, thus the valve actuator 60 and 60a will not be necessary in the second preferred embodiment.

### 3. Third Preferred Embodiment

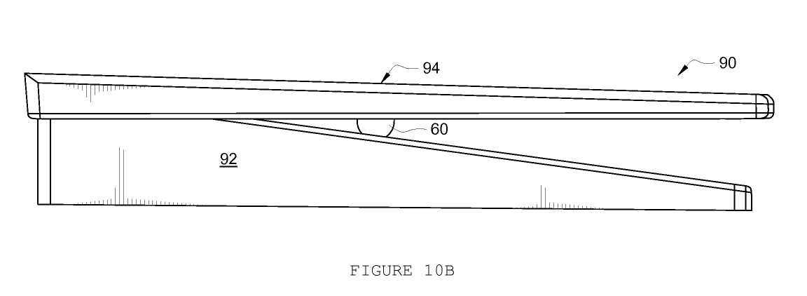

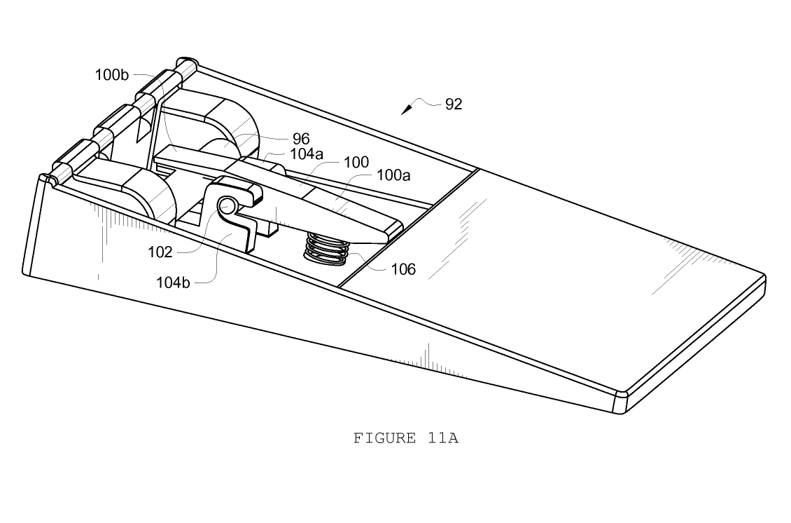

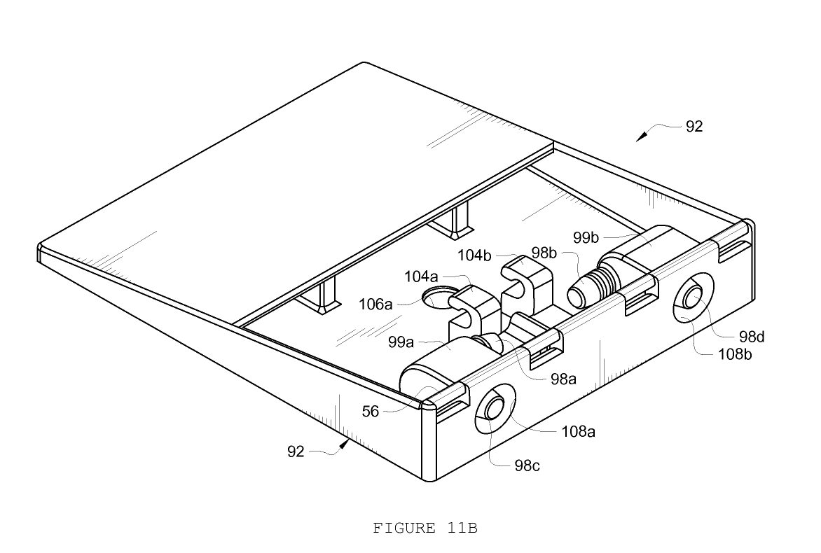

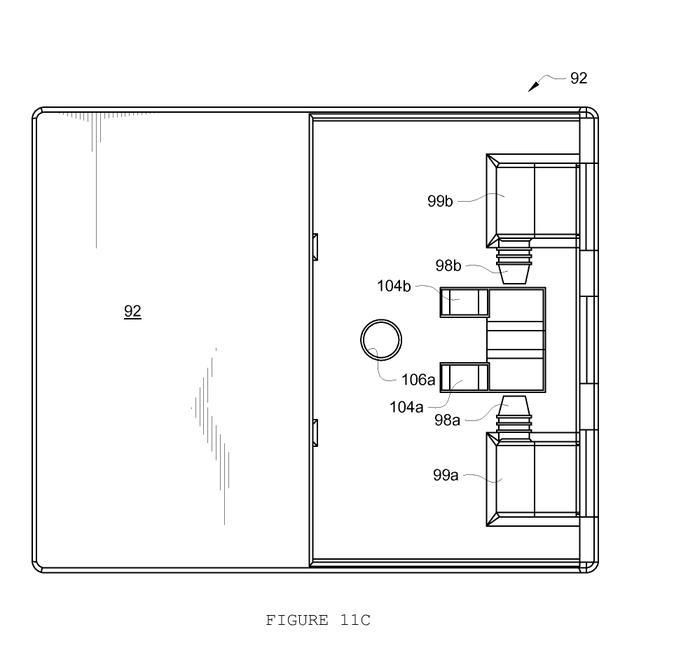

As shown in FIGS. 10A to 10B, 11A to 11C, the third preferred embodiment of the invention includes truncate tube 96, a lever 100 that functions to deform and close the truncate tube 96, and a spring 106 that functions to rotate and apply force to the lever 100 to close truncate tube 96 that are assembled as the fluid regulator 2, and a foot pedal 16 that functions to rotate the lever 100 away from the truncate tube 96, opening the truncate tube 96 and allowing flow. The third embodiment also includes tube mounts 98a and 98b as the OUT connection 13 and the IN connection 17, respectively.

As best understood in connection with FIGS. 11A to 11C, this embodiment eliminates the ball check valve of the first and second preferred embodiments. Truncate tube 96 is formed of a compressible, flexible and resilient material such as rubber, elastomeric plastic or the like and is attached at its opposite ends to tube mounts 98a, 98b (FIGS. 11B, 11C) and as depicted in FIG. 9A, tube 96 is pinched and normally closed by spring-loaded lever 100. Transversely disposed pin 102 is a part of or extends through lever 100 and the opposite ends of said pin are rotatably retained by transversely spaced apart retainers 104a and 104b that are integrally formed with the base 36 (further described below). Spring 106 or other suitable bias means provides constant upward pressure on the trailing end 100a of lever 100 so that leading end 100b of lever 100 bears downwardly against tube 96, thereby pinching it so that no liquid fluid can flow through said tube. A suitable, preferably bulbous, protrusion having a linear extent greater than the diameter of tube 96 is formed on the underside of leading end 100b to facilitate the pinching action.

The foot support 34 of the foot pedal 16 of the third preferred embodiment is similar or identical to that described in the first preferred embodiment. As best understood in connection with FIG. 10B, valve actuator 60 extends from the underside of foot pedal 94 and abuttingly engages trailing end 100a or lever 100 so that full depression of said foot support 94 lifts leading end 100b of lever 100 from truncate tube 96 so that liquid fluid may flow through. A physician controls the rate of fluid flow by manipulating the amount of compression of spring 106 thereby creating variable OPEN states.

The base 36 of the foot pedal 16 of the third preferred embodiment is similar to that described in the first preferred embodiment, further comprising retainers 104a, 104b (mentioned above), a shallow recess 106a (FIGS. 11B, 11C) is formed in a bottom wall of base 36 to accept a lower end of said spring, and tube mounts 98a and 98b.

Tube mounts 98a, 98b are housed in housings 99a, 99b and have a ninety degree (90.degree.) bend formed in them as best understood in connection with FIG. 11B. The respective ends 98c, 98d of said mounting tubes that do not engage truncate tube 96 are flush with the distal end of base 92 and are concentrically positioned within countersunk openings 108a, 108b. Inlet hose 18 is thus easily connected to tube mount 98c and outlet hose 14 is easily connected to tube mount 98d. Because the fluid regulator 2 of the third preferred embodiment allows fluid to pass through equally effectively in both forward and backward directions, the inlet hose 18 and outlet hose 14 may be mounted to the tube mounts 98c and 98d interchangeably.

As a person skilled in the art will recognize from the previous detailed description and from the figures and claims, modifications and changes can be made to the preferred embodiments of the invention without departing from the scope of this invention defined in the following claims.

We claim:

1. An arthroscopic surgery system comprising:
arthroscopic surgical tools;
a sterile fluid source;
a pump that pumps sterile fluid from the sterile fluid source to a surgical site;
a waste fluid collector; and
a controller including
an inlet that receives waste fluid from the surgical site,
an outlet that transmits the waste fluid to the waste fluid collector,
a fluid regulator normally in the CLOSED state,
a foot pedal that actuates the fluid regulator thereby switching the fluid regulator to the OPEN state.

2. The arthroscopic surgery system of claim 1 wherein the foot pedal includes a base, a foot support, a hinge pin that connects the base and the foot support, a spring that biases the foot pedal, a compression limiting element that prevents over-compression of the foot pedal, and an extension limiting element that prevents over-extension of the foot pedal.

3. The arthroscopic surgery system of claim 2 wherein the foot pedal further includes a friction surface on the foot support.

4. The arthroscopic surgery system of claim 2 wherein actuation of the fluid regulator is accomplished by an action selected from the group consisting of: compression of the foot pedal and extension of the foot pedal.

5. The arthroscopic surgery system of claim 4 wherein the fluid regulator includes a fluid regulator switch actuated by the foot pedal that electronically switches the fluid regulator to the OPEN state.

6. The arthroscopic surgery system of claim 5 wherein the fluid regulator switch is variable and the OPEN state of the fluid regulator is correspondingly variable.

7. The arthroscopic surgery system of claim 4 wherein the fluid regulator is mechanically actuated to the OPEN state.

8. The arthroscopic surgery system of claim 7 wherein the mechanical actuation is variable and the OPEN state of the fluid regulator is correspondingly variable.

9. The arthroscopic surgery system of claim 8 wherein the fluid regulator includes a pliable orifice that deforms by compression of the foot pedal and a sprung ball that blocks the pliable orifice at rest.

10. The arthroscopic surgery system of claim 8 wherein the fluid regulator includes a sprung post that moves under compression of the foot pedal and a disk connected to the sprung post and adapted to fully cover an orifice at rest.

11. The arthroscopic surgery system of claim 8 wherein the fluid regulator includes a lever that is hinged onto the base and is rotated away from a rest position by compression of the foot pedal, a spring that returns the lever to the rest position, and a pliable tube that is closed by the pressure of the lever and the spring at rest.

12. The arthroscopic surgery system of claim 2 wherein the device further comprises tubing, and wherein the foot pedal further includes an IN connection to the inlet of the fluid regulator and an OUT connection to the outlet of the fluid regulator.

13. The arthroscopic surgery system of claim 12 wherein the axis of the IN connection and the OUT connection are oriented parallel to each other and are perpendicular to the axis of the hinge pin.

14. The arthroscopic surgery system of claim 12 wherein the axis of the IN connection is collinear with the axis of the OUT connection and is parallel to the axis of the hinge pin.

15. The arthroscopic surgery system of claim 12 wherein the tubing includes an inlet tubing with one end that receives waste fluid from the surgical site and another end that connects to the IN connection, and the tubing includes an outlet tubing with one end that connects to the OUT connection and another end that connects with the waste fluid collector.

16. The arthroscopic surgery system of claim 12 wherein the foot pedal, the fluid regulator, and the tubing are disposable.

17. The arthroscopic surgery system of claim 12 wherein the foot pedal, the fluid regulator, and the tubing may be sterilized.

18. An arthroscopic surgery system that includes arthroscopic surgical tools, a sterile fluid source, a pump that pumps sterile fluid from the sterile fluid source to a surgical site, and a waste fluid collector, the improvement comprising a device that includes an inlet that receives waste fluid from the surgical site, an outlet that transmits the waste fluid to the waste fluid collector, a fluid regulator normally in the CLOSED state, and a foot pedal that actuates the fluid regulator, thereby switching the fluid regulator to the OPEN state.

19. The arthroscopic surgery system of claim 18, wherein the foot pedal includes a base, a foot support, a hinge pin that connects the base and the foot support, a spring that biases the foot pedal, a compression limiting element that prevents over-compression of the foot pedal, and an extension limiting element that prevents over-extension of the foot pedal.

20. A controller for an arthroscopic surgery system that includes arthroscopic surgical tools, a sterile fluid source, a pump that pumps sterile fluid from the sterile fluid source to a surgical site, and a waste fluid collector, the device comprising:
an inlet that receives waste fluid from the surgical site,
an outlet that transmits the waste fluid to the waste fluid collector,
a fluid regulator normally in the CLOSED state,
a foot pedal that actuates the fluid regulator, thereby switching the fluid regulator to the OPEN state.

21. The controller of claim 20 wherein the foot pedal includes a base, a foot support, a hinge pin that connects the base and the foot support, a spring that biases the foot pedal, a compression limiting element that prevents over-compression of the foot pedal, and an extension limiting element that prevents over-extension of the foot pedal.

22. The controller of claim 21 wherein the foot pedal further includes a friction surface on the foot support.

23. The controller of claim 21 wherein actuation of the fluid regulator is accomplished by an action selected from the group consisting of: compression of the foot pedal and extension of the foot pedal.

24. The controller of claim 23 wherein the fluid regulator includes a fluid regulator switch actuated by the foot pedal that electronically switches the fluid regulator to the OPEN state.

25. The controller of claim 24 wherein the fluid regulator switch is variable and the OPEN state of the fluid regulator is correspondingly variable.

26. The controller of claim 23 wherein the fluid regulator is mechanically actuated to the OPEN state.

27. The controller of claim 26 wherein the mechanical actuation is variable and the OPEN state of the fluid regulator is correspondingly variable.

28. The controller of claim 27 wherein the fluid regulator includes a pliable orifice that deforms by compression of the foot pedal and a sprung ball that blocks the pliable orifice at rest.

29. The controller of claim 27 wherein the fluid regulator includes a sprung post that moves under compression of the foot pedal and a disk that is connected to the sprung post and fully covers an orifice at rest.

30. The controller of claim 27 wherein the fluid regulator includes a lever that is hinged onto the base is rotated away from a rest position by compression of the foot pedal, a spring that returns the lever to the rest position, and a pliable tube that is closed by the pressure of the lever and the spring at rest.

31. The controller of claim 21 wherein the device further comprises tubing, and wherein the foot pedal further includes an IN connection to the inlet of the fluid regulator and an OUT connection to the outlet of the fluid regulator.

32. The controller of claim 31 wherein the axis of the IN connection and the OUT connection are oriented parallel to each other and are perpendicular to the axis of the hinge pin.

33. The controller of claim 31 wherein the axis of the IN connection is collinear with the axis of the OUT connection and is parallel to the axis of the hinge pin.

34. The controller of claim 20 wherein the tubing includes an inlet tubing with one end that receives waste fluid from the surgical site and another other end that connects to the IN connection, and the tubing includes an outlet tubing with one end that connects to the OUT connection and another end that connects with the waste fluid collector.

35. The controller of claim 34 wherein the foot pedal, the fluid regulator, and the tubing are disposable.

36. The controller of claim 34 wherein the foot pedal, the fluid regulator, and the tubing may be sterilized.

37. A method of selling a disposable foot pedal for arthroscopic surgery comprising the steps of:
providing a sterilized foot pedal;
providing a sterilized fluid regulator;
providing sterilized tubing;
packaging the foot pedal in a sterilized package;
packaging the fluid regulator in a sterilized package;
packaging the sterilized tubing in a sterilized package; and
selling the foot pedal, the fluid regulator, and the tubing together.

38. The method of claim 37 wherein the foot pedal and the fluid regulator are packaged into the same sterilized package.

39. The method of claim 38 wherein the foot pedal and the fluid regulator are pre-assembled and then packaged into the same sterilized package.

40. The method of claim 37 wherein the foot pedal, fluid regulator, and sterilized tubing are packaged into the same sterilized package.

41. The method of claim 40 wherein the foot pedal, fluid regulator, and sterilized tubing are pre-assembled and then packaged into the same sterilized package.

42. A method of arthroscopic surgery carried out by a surgeon comprising the steps of controlling arthroscopic surgical tools with a hand, controlling an arthroscopic camera with another hand, and controlling sterile fluid flow through the arthroscopic surgical site with a foot.
